(19) United States
(12) Patent Application Publication
(10) Pub. No.: US 2016/0220325 A1
(43) Pub. Date: Aug. 4, 2016

(54) ULTRASONIC PROBE AND ULTRASONIC APPARATUS HAVING THE SAME

(71) Applicant: SAMSUNG MEDISON CO., LTD., Gangwon-do (KR)

(72) Inventors: Nam Du JEON, Seoul (KR); Dong Gyu HYUN, Gwangju-si (KR)

(21) Appl. No.: 14/964,321

(22) Filed: Dec. 9, 2015

(30) Foreign Application Priority Data
Feb. 4, 2015 (KR) 10-2015-0017183

(51) Int. Cl. A61B 90/98; A61B 8/08; A61B 8/14; A61B 8/00

(52) U.S. Cl. CPC A61B 90/98; A61B 8/4444; A61B 8/5269; A61B 8/14; A61B 2562/222

(57) ABSTRACT

Disclosed is a ultrasonic probe provided with a plurality of RFID tags each including identification information, and a ultrasonic apparatus having the same. The ultrasonic probe includes a housing, a piezoelectric member receiving echo-ultrasonic waves reflected from a subject, and a plurality of radio-frequency identification (RFID) tags.

### CROSS-REFERENCE TO RELATED APPLICATION

This application claims the benefit of Korean Patent Application No. 10-2015-0017183, filed on Feb. 4, 2015, in the Korean Intellectual Property Office, the disclosure of which is incorporated herein by reference.

### BACKGROUND

1. Field

Embodiments of the present disclosure relate to a ultrasonic probe that transmits and receives ultrasonic waves to/from a subject, and a ultrasonic apparatus having the same.

2. Description of the Related Art

Ultrasonic apparatuses operate to irradiate ultrasonic waves through the surface of a subject to a target site inside a body and receive echo ultrasonic waves reflected from the target site to obtain a cross-sectional image of a soft tissue or bloodstream by using information about the echo ultrasonic waves in a non-invasive manner.

A ultrasonic probe forming the ultrasonic apparatus irradiates ultrasonic waves to a subject and receives echo-ultrasonic waves reflected from the subject. In detail, the ultrasonic probe operates to convert an electric signal to ultrasonic waves, irradiate the converted ultrasonic waves to a subject, receive echo-ultrasonic waves reflected from the subject, convert the received echo-ultrasonic waves into an echo-ultrasonic signal, and transmit the echo-ultrasonic signal to a main body of the ultrasonic apparatus.

The ultrasonic apparatus is provided in various types depending on the uses or shapes. A user may select a desired type of ultrasonic probe in consideration of a condition of a region of a subject to be diagnosed. In this case, the ultrasonic probe selected by a user is provided with a radio-frequency identification (RFID) tag including identification information such that the main body can easily identify the ultrasonic probe.

### SUMMARY

Therefore, it is an aspect of the present disclosure to provide a ultrasonic probe provided with a plurality of RFID tags each including identification information, and a ultrasonic apparatus having the same.

In accordance with an embodiment of the present disclosure, a ultrasonic probe includes a housing and a plurality of radio-frequency identification (RFID) tags.

The RFID tag may include identification information about the ultrasonic probe.

The ultrasonic probe may further include a piezoelectric member configured to receive echo-ultrasonic waves reflected from a subject.

The ultrasonic probe may further include a shielding film provided inside the housing to block the received echo-ultrasonic waves from external noise. The RFID tag is provided in plural number to prevent a recognition rate of the RFID tag from being lowered due to the shielding film.

The RFID tag may include a recognition surface provided with an antenna pattern that is configured to transmit the identification information upon receiving a signal to request the identification information.

At least two of the plurality of RFID tags may have the recognition surfaces, respectively, which are oriented in directions different from each other.

At least two of the plurality of RFID tags may have the recognition surfaces, respectively, which are oriented in directions opposite to each other.

At least two of the plurality of RFID tags may have the recognition surfaces, respectively, which are perpendicular to each other.

At least one of the plurality of RFID tags may be detachably provided on a surface of the housing.

At least one of the plurality of RFID tags may be provided between the housing and the shielding film.

The ultrasonic probe may further include: an external cable configured to connect the ultrasonic probe to an external device; a strain relief configured to fix the external cable to the housing; and an internal cable configured to connect the external cable to the piezoelectric member.

At least one of the plurality of RFID tags may be provided at one of the external cable, the strain relief and the internal cable.

The identification information may include at least one of a name, a type, a manufacturer, a manufacturing date, and a serial number of the ultrasonic probe, and an identification number determined by a user.

In accordance with another embodiment of the present disclosure, a ultrasonic apparatus includes a radio-frequency identification (RFID) reader, a ultrasonic probe and a controller. The radio-frequency identification (RFID) reader may be configured to recognize an RFID tag. The ultrasonic probe may be provided with the RFID tag including identification information. The controller may be configured to, if the RFID reader recognizes the RFID tag provided at the ultrasonic probe, recognize the ultrasonic probe corresponding to the identification information of the recognized RFID tag, wherein the ultrasonic probe may be provided with a plurality of the RFID tags.

The RFID tag may include a recognition surface provided with an antenna pattern that is configured to transmit the identification information to the RFID reader upon receiving a signal to request the identification information from the RFID reader.

At least two of the plurality of RFID tags may have the recognition surfaces, respectively, which are oriented in directions different from each other.

At least two of the plurality of RFID tags may have the recognition surfaces, respectively, which are oriented in directions opposite to each other.

At least two of the plurality of RFID tags may have the recognition surfaces, respectively, which are perpendicular to each other.

The ultrasonic apparatus may further include a housing a piezoelectric member and a shielding film. The piezoelectric member may be configured to receive echo-ultrasonic waves reflected from a subject. The shielding film may be provided inside the housing to block the received echo-ultrasonic waves from external noise.

At least one of the RFID tags may be detachably provided on a surface of the housing.

At least one of the plurality of RFID tags may be provided between the housing and the shielding film.

The ultrasonic probe may include: an external cable connecting the ultrasonic probe to an external device; a strain relief fixing the external cable to the housing; and an internal cable connecting the external cable to the piezoelectric member.

At least one of the plurality of RFID tags may be provided at one of the external cable, the strain relief and the internal cable.

The identification information may include at least one of a name, a type, a manufacturer, a manufacturing date, a serial number of the ultrasonic probe, and an identification number determined by a user.

### BRIEF DESCRIPTION OF THE DRAWINGS

These and/or other aspects of the disclosure will become apparent and more readily appreciated from the following description of the embodiments, taken in conjunction with the accompanying drawings of which:

FIG. 1 is a view illustrating an external appearance of a ultrasonic apparatus in accordance with an embodiment of the present disclosure;

FIGS. 2A and 2B are control block diagrams illustrating ultrasonic apparatuses in accordance with embodiments of the present disclosure;

FIGS. 3A to 3c are views illustrating the interior of an ultrasonic probe;

FIGS. 4A and 4B are views illustrating ultrasonic probes in accordance with embodiments of the present disclosure, in which two RFID tags are provided on a surface of a housing;

FIGS. 5A and 5B are views illustrating a ultrasonic probe in which three RFID tags are provided on a surface of a housing and a ultrasonic probe in which four RFID tags are provided on a surface of a housing in accordance with embodiments of the present disclosure;

FIGS. 6A and 6B are views illustrating ultrasonic probes in accordance with embodiments of the present disclosure, in which two RFID tags are provided on a strain relief or an external cable;

FIGS. 7A and 7B are views illustrating ultrasonic probes in accordance with embodiments of the present disclosure, in which two RFID tags are provided inside a housing;

FIGS. 8A and 8B are views illustrating a ultrasonic probe in which three RFID tags are provided inside a housing, and a ultrasonic probe in which four RFID tags are provided inside a housing in accordance with embodiments of the present disclosure; and

FIG. 9 is a view illustrating an ultrasonic probe in accordance with an embodiment of the present disclosure, in which RFID tags are provided in/on a housing.

### DETAILED DESCRIPTION

Reference will now be made in detail to the embodiments of the present disclosure, examples of which are illustrated in the accompanying drawings, wherein like reference numerals refer to like elements throughout.

The terminology ‘a ultrasonic image’ used herein represents an image about a subject obtained by using ultrasonic waves. In addition, a ‘subject’ may represent human, animals, metal, non-metal or a part thereof. For example, the subject may include organs, such as a liver, a heart, a womb, a brain, breasts, and an abdomen, or a blood vessel. In addition, the subject may include a phantom. The phantom represents a substance having a volume that is highly approximate to the density and the effective atomic number of an organism.

Although the terminology ‘a user’ used herein may represent a medical professional, for example, a surgeon, a nurse, a medical laboratory technologist, and a medical imaging specialist, and may include a technician who fixes medical instrument, the present disclosure is not limited.

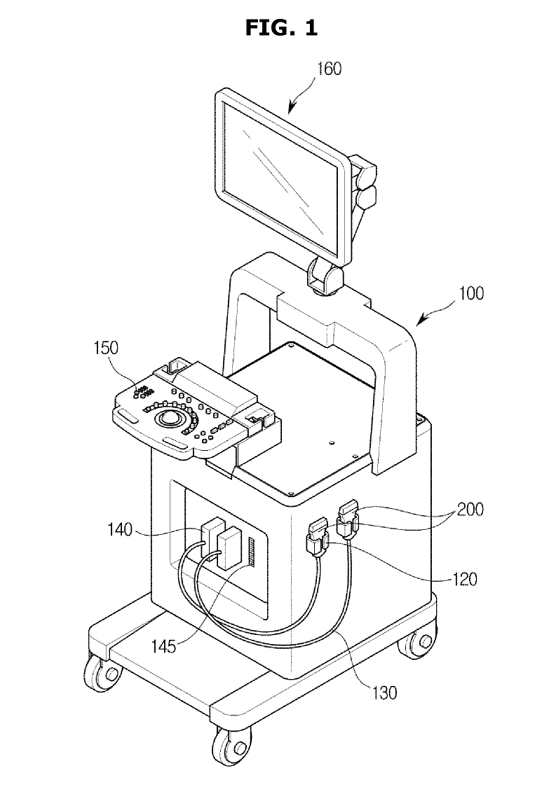

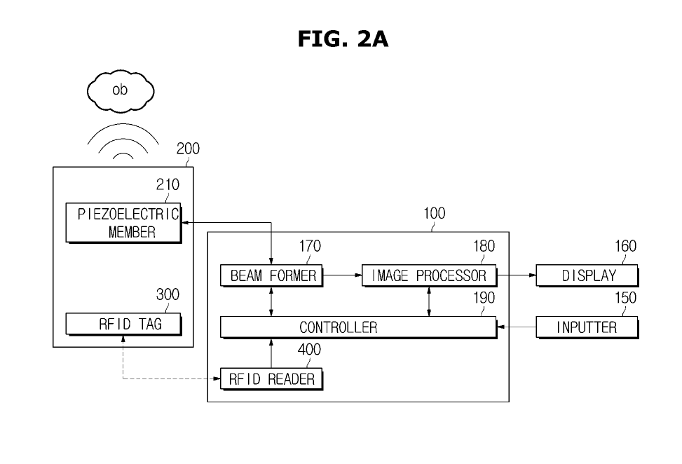

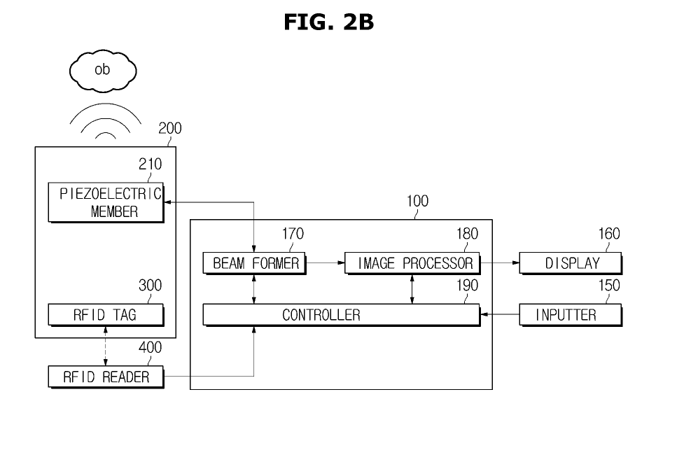

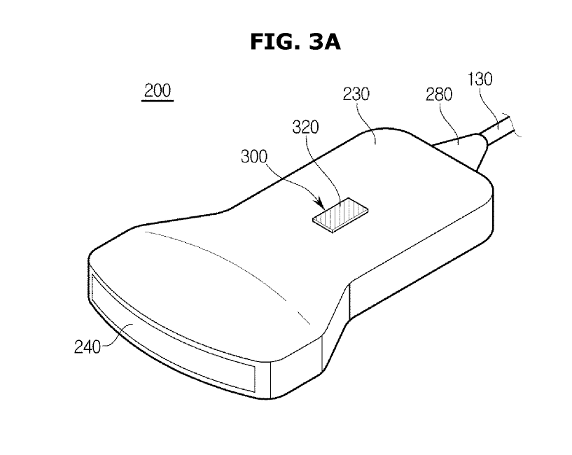

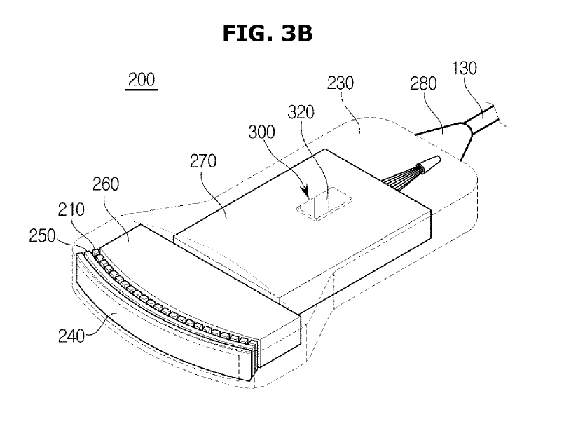

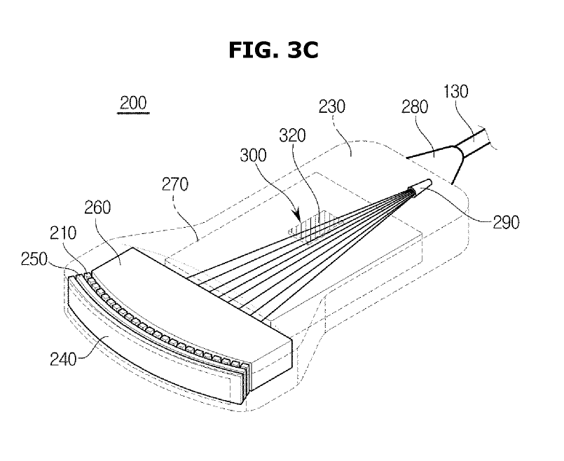

FIG. 1 is a view illustrating an external appearance of a ultrasonic apparatus in accordance with an embodiment of the present disclosure, FIGS. 2A and 2B are control block diagrams illustrating ultrasonic apparatuses in accordance with embodiments of the present disclosure, and FIGS. 3A to 3C are views illustrating interior of an ultrasonic probe.

Referring to FIG. 1, an ultrasonic apparatus includes a main body 100, a display 160 connected to the main body 100, an inputter 150 and an ultrasonic probe 200.

The main body 100 transmits an ultrasonic signal to the ultrasonic probe 200, receives an echo-ultrasonic signal from the ultrasonic probe 200, and based on the received echo-ultrasonic signal, generates an ultrasonic image.

To this end, the main body 100 includes a beam former 170 that performs beamforming to focus ultrasonic waves transmitted and received from and to the ultrasonic probe 200 respectively, an image processor 180 that generates ultrasonic images based on the beamformed ultrasonic echo signals, and a controller 190 that performs overall operation of the ultrasonic apparatus including the main body 100.

The beamformer 170 may perform beamforming to focus ultrasonic waves. The beamforming may include a transmission beamforming that delays ultrasonic waves irradiated at a certain point of an object ob so as to be arranged, and a reception beamforming that delays ultrasonic waves reflected from a certain point of an object ob so as to be arranged. When ultrasonic waves are transmitted to a certain position of the object ob there is time difference between the ultrasonic waves arriving at the certain position, and when ultrasonic waves are received from a certain position of an object ob to the ultrasonic probe 200, there is time difference between the ultrasonic waves arriving at the ultrasonic probe 200. Accordingly, the time difference is compensated through the beamforming.

The beamformer 170 may adopt a generally known beamforming method, and may apply a plurality of beamforming methods, or selectively apply a plurality of beamforms.

The image processor 180 may generate an ultrasonic image by processing the beamformed echo ultrasonic signals. The image processor 180 may process the echo ultrasonic signals according to a generally known image processing method.

For example, the image processor 180 may perform a time gain compensation (TGC) on the beamformed echo ultrasonic signals. Thereafter, the image processor 180 may set a dynamic range (DR). After setting the DR, the image processor 180 may compress echo ultrasonic signals within the set dynamic range. Finally, the image processor 180 rectifies the echo ultrasonic signals, and removes noise from the rectified signals.

By using the echo ultrasonic signals processed as the above, the image processor 180 may generate an ultrasonic image. The image processor 180 may generate various types of ultrasonic images. Examples of the ultrasonic image generated by the image processor 180 may include an amplitude mode (A-mode) image, a brightness mode (B-mode), a motion mode (M-mode) image, and a Doppler mode image.

The ultrasonic image generated as the above may be provided to a user through the display 160. A user may visually check a ultrasonic image of an inside of an object ob provided through the display, thereby diagnosing an object ob, that is, a patient.

In addition, the display 160 may display various user interfaces (Uls) related to controlling the ultrasonic apparatus. A user may check the UI provided through the display 160, and input a command to control each component of the ultrasonic apparatus.

The display 160 may be implemented using one of a cathode ray tube (CRT) and a liquid crystal display (LCD). In addition, the display 160 may provide a three-dimensional image as well as a two-dimensional image.

The controller 190 may control the beamformer 170, the image processing apparatus 300 and/or the display 160 to control the overall operation of the ultrasonic apparatus. In addition, the controller 190 may control each component of the ultrasonic apparatus according to a control command received by the inputter 150 from a user.

For example, the controller 190 may control a beamforming method of the beamformer 170, and the image processing apparatus 300 may control a method of generating a ultrasonic image, and the display 160 may control a method of displaying a ultrasonic image.

In addition, when the ultrasonic probe 200 is provided in plural, and one of the plurality of ultrasonic probes 200 is selected by a user, the controller 190 may control the selected ultrasonic probe 200 to radiate ultrasonic waves. Details thereof will be described later.

The ultrasonic probe 200 is connected to one end of an external cable 130, and the other end of the external cable 130 may be connected to a male connector 140. The male connector 140 connected to the other end of the external cable 130 may be physically coupled to a female connector 145 of the main body 100.

The ultrasonic probe 200 may be integrally provided with the external cable 130, or may be detachabley provided from the external cable 130.

In addition, the ultrasonic probe 200 may further include a strain relief 280 configured to fix the external cable 130 to a housing 230 of the ultrasonic probe 200. A strain relief 280 protects the external cable 130 against external shock, and prevents the external cable 130 from being bent.

As described above, one ultrasonic probe 200 may be connected to one main body 100. In addition, a plurality of ultrasonic probes 200 may be connected to one main body 100 in a same manner as connecting one ultrasonic probe to one main body, and to this end, the main body 100 may include a plurality of female connectors. FIG. 1 illustrates an example in which two ultrasonic probes 200 are connected to one main body 100.

Alternatively, different from FIG. 1, the ultrasonic probe 200 may be connected to the main body 100 in a wireless manner. In this case, the ultrasonic probe 200 may transmit an echo-ultrasonic signal corresponding to echo-ultrasonic waves received from the object ob to the main body 100 in a wireless manner.

The ultrasonic probe 200 may transmit and receive ultrasonic waves to and from the object ob respectively, while making contact with the surface of the object ob. In detail, the ultrasonic probe 200 irradiates ultrasonic waves to the inside of the object ob according to an ultrasonic signal, which is an electric signal, provided from the main body 100, receives echo-ultrasonic waves reflected from a specific portion, and transmits an echo-ultrasonic signal corresponding to the echo ultrasonic waves to the main body 100.

To this end, the ultrasonic probe 200 may include a piezoelectric member 210.

The piezoelectric member 210 may include a plurality of elements that vibrate to execute conversion between an electric signal and ultrasonic waves. The plurality of elements may be arranged on one surface of the housing 230 of the ultrasonic probe 200. The plurality of elements are arranged on one surface of the housing 230 of the ultrasonic probe 200. In detail, a plurality of piezoelectric members 210 are arranged in parallel to an opening formed through one surface of the housing 230 such that transmission/reception of ultrasonic waves is performed through the opening.

Referring to FIGS. 3A and 3C, the opening formed through one surface of the housing 230 is provided with a lens 240 allowing ultrasonic waves to pass therethrough. The lens 240 may divide inside from outside of the housing 230 without interfering with ultrasonic waves from travelling. In addition, the lens 240 serves to collect the irradiated ultrasonic waves according to a predetermined curvature of the lens.

A matching layer 250 is provided on one surface of the lens 240 to match acoustic impedance. The piezoelectric member 210 having a plurality of elements is provided on one surface of the matching layer 250. In addition, a backing layer 260 is formed on one surface of the piezoelectric member 210 to absorb ultrasonic waves generating distortion of image.

The ultrasonic probe 200 may further include an internal cable 290 connecting the piezoelectric member 210 to the external cable 130. The internal cable 290 may transmit an ultrasonic signal transmitted from the main body 100 through the external cable 130 to the piezoelectric member 210, or transmit an echo-ultrasonic signal to the main body 100 through the external cable 130.

In this case, the internal cable 290 may be shielded by the shielding film 270 against noise introduced from outside. The internal cable 290 is passed by an echo-ultrasonic signal based on which an ultrasonic image is generated, and the shielding film 270 prevents distortion of an ultrasonic image by shielding the echo-ultrasonic signal against noise.

The shielding film 270 may be formed using metal in order to prevent external noise from being introduced to the inside of the ultrasonic probe 200.

Meanwhile, the ultrasonic apparatus is designed to irradiate ultrasonic waves through the ultrasonic probe 200 only at the time of ultrasonic diagnosis. In particular, when a plurality of ultrasonic probes 200 are connected to the main body 100, only a desired one of the ultrasonic probes 200 needs to be controlled to radiate ultrasonic waves.

To this end, the inputter 150 may receive a command to select a ultrasonic probe 200 desired to radiate. If a user selects a desired ultrasonic probe 200 through the inputter 150 prior to a ultrasonic diagnosis, the controller 190 may control the selected ultrasonic probe 200 to radiate ultrasonic waves.

Such a method of inputting a command to select the ultrasonic probe 200 may cause inconvenience to a user, and takes time.

In order to remove such a constraint, the ultrasonic probe 200 may include an RFID tag 300. The RFID tag 300 may include identification information about the ultrasonic probe 200. The identification information represents information for identifying each of the plurality of ultrasonic probes 200 connected to the main body 100. The identification information may include at least one of a name, a type, a manufacturer, a manufacturing date, and a serial number of the ultrasonic probe, and an identification number determined by a user.

Referring to FIG. 2A, the main body 100 may include an RFID reader 400. Alternatively, referring to FIG. 2B, an RFID reader 400 may be separately provided from the main body 100.

The RFID reader 400 recognizes the RFID tag 300, and transmits identification information about the ultrasonic probe 200 included in the RFID tag 300 to the controller 190. The controller 190 checks the identification information and recognizes the ultrasonic probe 200 corresponding to the identification information, and controls the recognized ultrasonic probe 200 to irradiate ultrasonic waves.

However, referring to FIG. 3B, the shielding film 270 is provided inside the housing 230, and may hinder the RFID reader 400 from recognizing the RFID tag 300.

In general, the RFID reader 400 transmits an identification information request signal within a preset distance. If the RFID tag 300 is positioned within the present distance, the RFID tag 300 may receive the identification information request signal and transmit an identification information signal corresponding to the identification information request signal to the RFID reader 400. Such a series of processes of recognizing identification information by the RFID reader 400 is referred to as a recognition process of the RFID tag 300.

In order for the RFID reader 400 to perform the recognition process, the RFID tag 300 may include a recognition surface 320 on which an antenna pattern is formed to receive an identification information request signal and transmit an identification information signal corresponding to the identification information request signal. To this, the recognition surface may be provided as a surface having the highest recognition rate among a plurality of surfaces forming the RFID tag 300.

In this case, when the identification information request signal or the identification information signal are blocked from travelling by the shielding film 270, the recognition rate the RFID tag 300 by the RFID reader 400 is lowered.

Table 1 shows an experiment result related to the recognition rate, in which the number of times by which the RFID reader 400 recognizes the RFID tag 300 for 5 secs is checked by using the ultrasonic probe 200 provided with the RFID tag 300 attached to one surface of the housing 230 thereof. The number of recognition times of the RFID tag 300 is checked while changing the distance between an antenna of the RFID reader 400 and the RFID tag 300, a tagging direction (defined based on surface A to which a RFID tag is attached and surface B provided on the opposite side of surface A) and the number of tags.

TABLE 1

| | | | Tagging | Number of tag recognition |
|---|---|---|---|---|
| No. | distance | Number of tags | direction | times |
| 1 | 1 cm | 1 | Surface A | 143 |
| | | | Surface B | 149 |
| 2 | 5 cm | 1 | Surface A | 145 |
| | | | Surface B | 0 |

Referring to Table 1, when the distance between the RFID antenna and the RF tag 300 is 1 cm, each of surface A and surface B has a high recognition rate. However, when the distance between the RFID antenna and the RF tag 300 is 5 cm, the RFID tag 300 is not recognized in the direction of surface B.

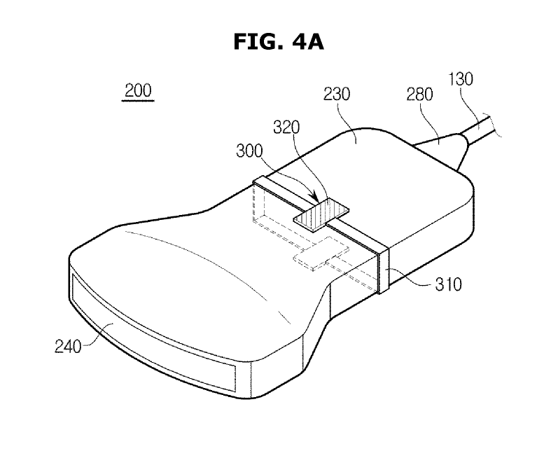

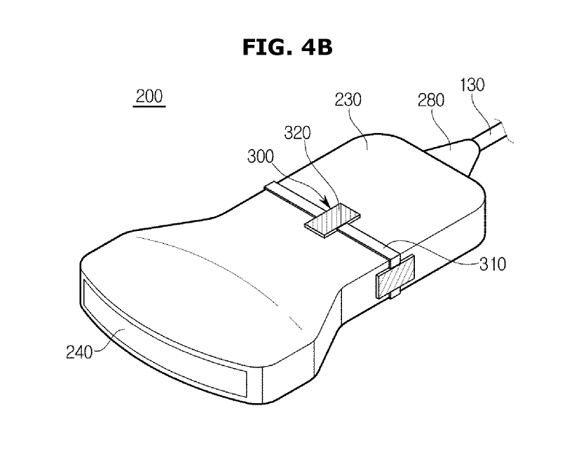

Accordingly, in order to prevent the recognition rate from being lowered due to the shield film 270, the ultrasonic probe 200 includes a plurality of RFID tags 300. Hereinafter, the ultrasonic probe 300 having a plurality of RFID tags 300 will be described with reference to FIGS. 4 to 8.

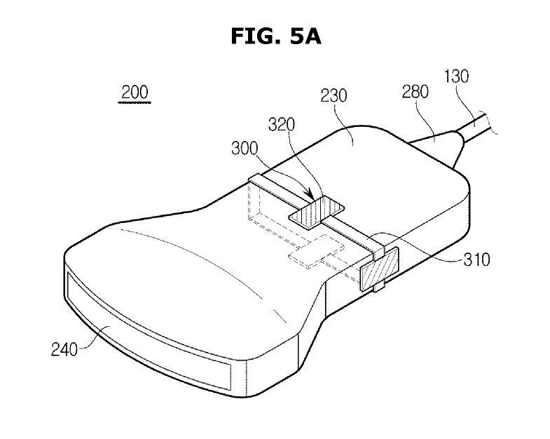

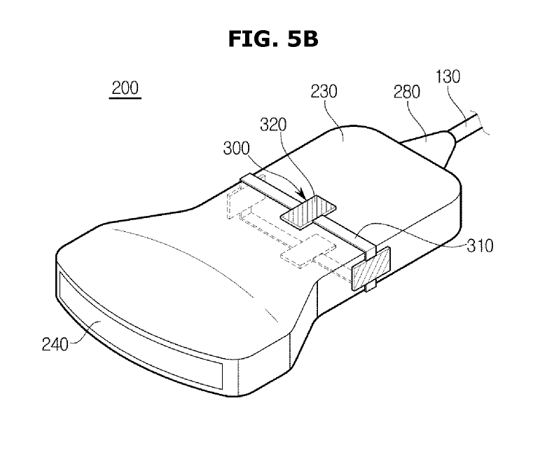

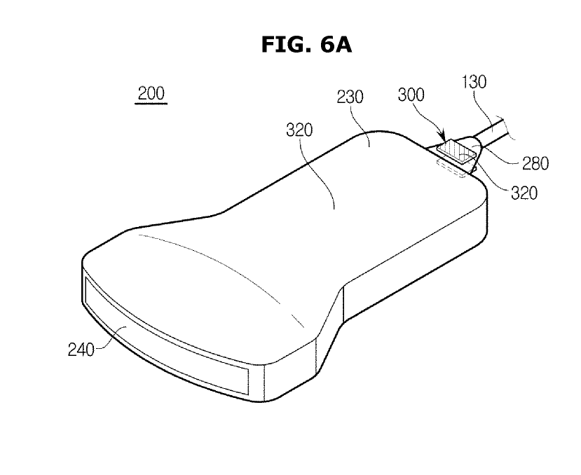

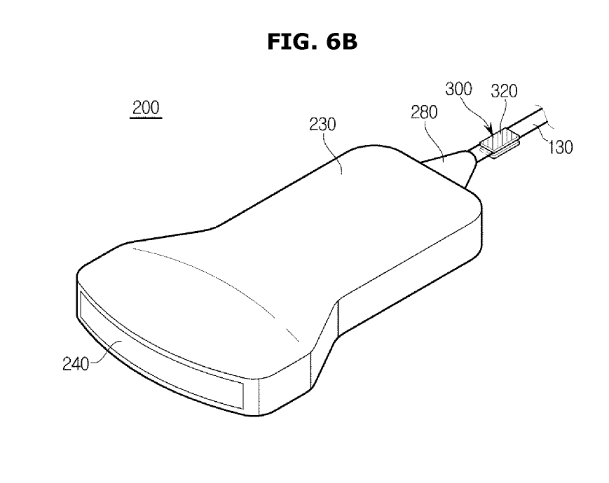

FIGS. 4A and 4B illustrate an ultrasonic probe in accordance with embodiments of the present disclosure, in which two RFID tags are provided on a surface of a housing, FIGS. 5A and 5B illustrate a ultrasonic probe in which three RFID tags are provided on a surface of a housing and a ultrasonic probe in which four RFID tags are provided on a surface of a housing in accordance with embodiments of the present disclosure, and FIGS. 6A and 6B are views illustrating ultrasonic probes in accordance with embodiments of the present disclosure, in which two RFID tags are provided on a strain relief or an external cable.

At least two of a plurality of RFID tags 300 provided on the ultrasonic probe 200 may have recognition surfaces 320, respectively, which are oriented in different directions. As a result, the recognition rate of the RFID reader 400 may be improved in various directions.

Referring to FIG. 4A, the recognition surfaces 320 of the two RFID tags 300 are provided to be oriented in directions opposite to each other. Alternatively, referring to FIG. 4B, the recognition surfaces 320 of the two RFID tags 300 are provided to be perpendicular to each other.

Referring to FIGS. 5A and 5B, recognition surfaces 320 of a plurality of RFID tags 300 may be provided to be oriented in directions opposite to each other, or perpendicular to each other.

The plurality of RFID tags 300 may be detachably provided on the surface of the housing 230 of the ultrasonic probe 200. For example, the plurality of RFID tags 300 may be provided with a band 310. The plurality of RFID tags 300 may be fixed to the surface of the housing 230 of the ultrasonic probe 200 through the band 310.

The plurality of RFID tags 300 may be attached to a surface of the strain relief 280 or a surface of the external cable 130 as well as the housing 230. FIG. 6A illustrates a case in which the two RFID tags 300 are attached to the strain relief 280, and FIG. 6B illustrates a case in which the two RFID tags 300 are attached to the external cable 130. Similarly, the two RFID tags 300 may have recognition surfaces, respectively, which are directed in different directions from each other.

As described above, the plurality of RFID tag 300 is detachably provided on the surface of the ultrasonic probe 200, and thus easily attached to the complete ultrasonic probe 200.

The above description has been made in relation to the plurality of RFID tags 300 that are detachably provided. Different from the above, the plurality of RFID tags 300 may be provided inside the housing 230. The following description will be made in relation to the RFID tags 300 provided inside the housing 230.

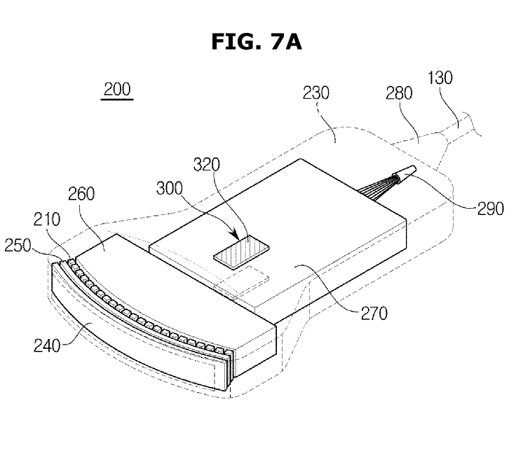

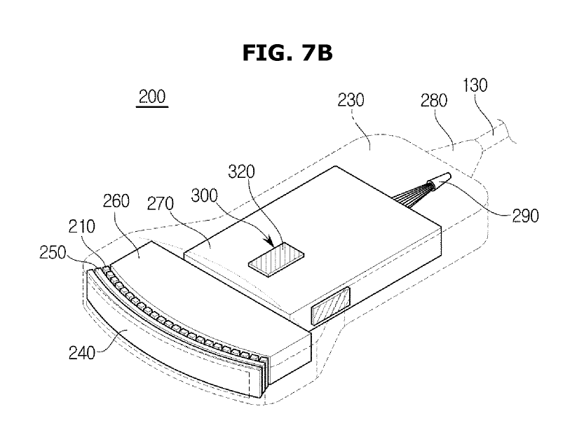

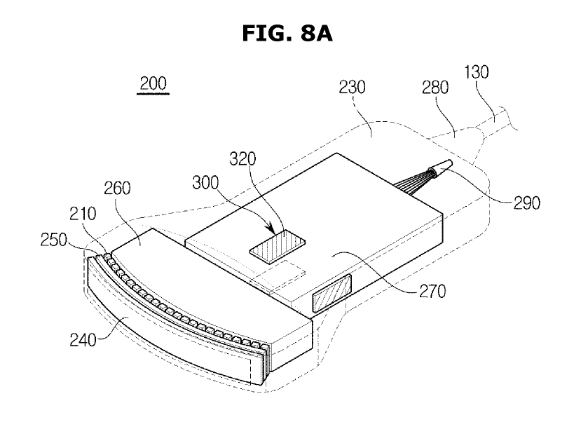

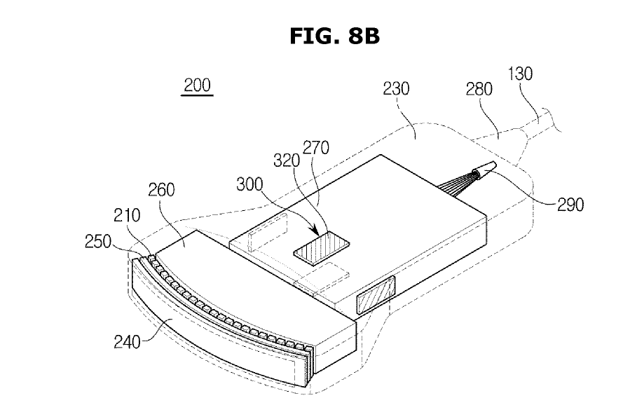

FIGS. 7A and 7B are views illustrating ultrasonic probes in accordance with embodiments of the present disclosure, in which two RFID tags are provided inside a housing, and FIGS. 8A and 8B are views illustrating a ultrasonic probe in which three RFID tags are provided inside a housing, and a ultrasonic probe in which four RFID tags are provided inside a housing in accordance with embodiments of the present disclosure.

The plurality of RFID tags 300 may be provided inside the housing 230, in detail, between the housing 230 and the shielding film 270. For example, the plurality of RFID tags 300 may be attached to a surface of the shielding film 270 or may be attached to the internal cable 290 inside the housing 230.

As described above, the position in which the RFID tags 300 are provided in the housing 230 is not limited as long as the plurality of RFID tags 300 have the recognition surfaces 320 oriented in different directions from each other.

On FIG. 7A, the recognition surfaces 320 of the two RFID tags 300 are provided to be oriented in opposite directions. On the contrary, FIG. 7B illustrates that the recognition surfaces 320 of the two RFID tags 300 are provided to be perpendicular to each other.

In addition, referring to FIGS. 8A and 8B, the recognition surfaces 320 of the plurality of RFID tags 300 are provided to be oriented in opposite directions, or provided to be perpendicular to each other.

For the RFID tags provided inside the housing 230, the ultrasonic probe 200 needs to have the RFID tags formed at a fixed position, so that the ultrasonic probe 200 may perform a stable identification.

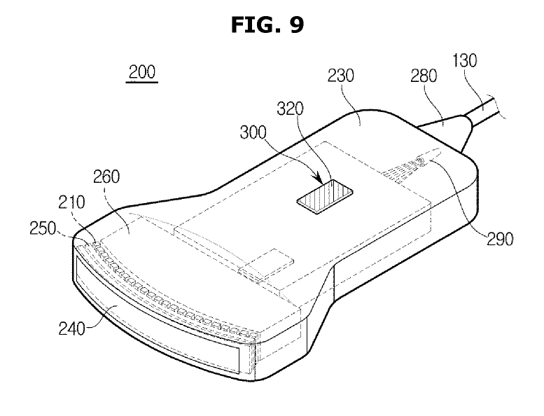

FIG. 9 is a view illustrating an ultrasonic probe in accordance with an embodiment of the present disclosure, in which RFID tags are provided both at the inside and the surface of a housing.

Different from the description of FIGS. 4 to 8, the RFID tag 300 may be provided both at the inside and the surface of the housing 230. FIG. 9 illustrates that one RFID tag 300 is provided on the surface of the housing 230, and another RFID tag 300 is provided between the housing 230 and the shielding film 270.

As described above, the positions of the RFID tags 300 are not limited as long as the plurality of RFID tags 300 have the recognition surfaces 320 oriented in different directions from each other so that the recognition rate of the RFID reader 400 is improved.

As described above, the plurality of RFID tags 300 are provided at the ultrasonic probe 200, so that the ultrasonic probe 200 may be easily recognized by the RFID reader 400 regardless of the tagging direction. As a result, identification information about a desired ultrasonic probe 200 among the plurality of ultrasonic probes 200 may be easily checked and controlled.

As is apparent from the above, the disclosed ultrasonic probe and the ultrasonic apparatus having the same can improve an RFID tag recognition rate for identifying the ultrasonic probe.

Although a few embodiments of the present disclosure have been shown and described, it would be appreciated by those skilled in the art that changes may be made in these embodiments without departing from the principles and spirit of the disclosure, the scope of which is defined in the claims and their equivalents.

What is claimed is:

1. A ultrasonic probe comprising:
a housing; and
a plurality of radio-frequency identification (RFID) tags.

2. The ultrasonic probe of claim 1, wherein the RFID tag includes identification information about the ultrasonic probe.

3. The ultrasonic probe of claim 1, further comprising a piezoelectric member configured to receive echo-ultrasonic waves reflected from a subject.

4. The ultrasonic probe of claim 3, wherein the ultrasonic probe further comprises a shielding film provided inside the housing to block the received echo-ultrasonic waves from external noise;
the RFID tag is provided in plural number to prevent a recognition rate of the RFID tag from being lowered due to the shielding film.

5. The ultrasonic probe of claim 1, wherein the RFID tag includes a recognition surface provided with an antenna pattern that is configured to transmit the identification information upon receiving a signal to request the identification information.

6. The ultrasonic probe of claim 5, wherein at least two of the plurality of RFID tags have the recognition surfaces, respectively, which are oriented in directions different from each other.

7. The ultrasonic probe of claim 6, wherein at least two of the plurality of RFID tags have the recognition surfaces, respectively, which are oriented in directions opposite to each other.

8. The ultrasonic probe of claim 6, wherein at least two of the plurality of RFID tags have the recognition surfaces, respectively, which are perpendicular to each other.

9. The ultrasonic probe of claim 1, wherein at least one of the plurality of RFID tags is detachably provided on a surface of the housing.

10. The ultrasonic probe of claim 4, wherein at least one of the plurality of RFID tags is provided between the housing and the shielding film.

11. The ultrasonic probe of claim 3, further comprising:
an external cable configured to connect the ultrasonic probe to an external device;
a strain relief configured to fix the external cable to the housing; and
an internal cable configured to connect the external cable to the piezoelectric member.

12. The ultrasonic probe of claim 11, wherein at least one of the plurality of RFID tags is provided at one of the external cable, the strain relief and the internal cable.

13. The ultrasonic probe of claim 1, wherein the identification information includes at least one of a name, a type, a manufacturer, a manufacturing date, and a serial number of the ultrasonic probe, and an identification number determined by a user.

14. A ultrasonic apparatus comprising:
a radio-frequency identification (RFID) reader configured to recognize an RFID tag;
a ultrasonic probe provided with the RFID tag including identification information; and
a controller configured to, if the RFID reader recognizes the RFID tag provided at the ultrasonic probe, recognize the ultrasonic probe corresponding to the identification information of the recognized RFID tag,
wherein the ultrasonic probe is provided with a plurality of the RFID tags.

15. The ultrasonic apparatus of claim 14, wherein the RFID tag includes a recognition surface provided with an antenna pattern that is configured to transmit the identification information to the RFID reader upon receiving a signal to request the identification information from the RFID reader.

16. The ultrasonic apparatus of claim 15, wherein at least two of the plurality of RFID tags have the recognition surfaces, respectively, which are oriented in directions different from each other.

17. The ultrasonic apparatus of claim 16, wherein at least two of the plurality of RFID tags have the recognition surfaces, respectively, which are oriented in directions opposite to each other.

18. The ultrasonic apparatus of claim 16, wherein at least two of the plurality of RFID tags have the recognition surfaces, respectively, which are perpendicular to each other.

19. The ultrasonic apparatus of claim 14, further comprising:
a housing;
a piezoelectric member configured to receive echo-ultrasonic waves reflected from a subject; and
a shielding film provided inside the housing to block the received echo-ultrasonic waves from external noise.

20. The ultrasonic apparatus of claim 19, wherein at least one of the RFID tags is detachably provided on a surface of the housing.

21. The ultrasonic apparatus of claim 19, wherein at least one of the plurality of RFID tags is provided between the housing and the shielding film.

22. The ultrasonic apparatus of claim 19, wherein the ultrasonic probe comprises:
an external cable connecting the ultrasonic probe to an external device;
a strain relief fixing the external cable to the housing; and
an internal cable connecting the external cable to the piezoelectric member.

23. The ultrasonic apparatus of claim 22, wherein at least one of the plurality of RFID tags is provided at one of the external cable, the strain relief and the internal cable.

24. The ultrasonic apparatus of claim 14, wherein the identification information includes at least one of a name, a type, a manufacturer, a manufacturing date, a serial number of the ultrasonic probe, and an identification number determined by a user.
